(19) United States
(12) Patent Application Publication
(10) Pub. No.: US 2026/0250224 A1
(43) Pub. Date: Aug. 27, 2026

(54) PROCESS FOR SYNTHESIZING PROPOFOL BY CONTINUOUS FLOW CHEMISTRY

(71) Applicant: TCG GREENCHEM INC., Ewing, NJ (US)

(72) Inventors: Maksim VASILEV, Levittown, PA (US); Aravind GANGU, Ewing, NJ (US); Chris SENANAYAKE, Brookfield, CT (US); Gopal SIRASANI, Pennington, NJ (US); Bo QU, Brookfield, CT (US)

(73) Assignee: TCG GREENCHEM INC., Ewing, NJ (US)

(21) Appl. No.: 19/549,155

(22) Filed: Feb. 25, 2026

Related U.S. Application Data
(60) Provisional application No. 63/763,364, filed on Feb. 26, 2025.

(51) Int. Cl. C07C 37/16

(52) U.S. Cl. CPC C07C 37/16

(57) ABSTRACT

The present invention relates to a continuous process for the preparation of a highly pure compound of formula III i.e. propofol, suitable for industrial or pharmaceutical use:

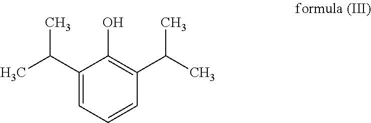

### REFERENCE TO RELATED APPLICATIONS

This application claims the benefit of U.S. Provisional Application Ser. No. 63/763,364, filed on Feb. 26, 2025; the disclosure of which is incorporated herein by reference.

### FIELD OF INVENTION

The present invention relates to a continuous process for the preparation of highly pure 2,6-diisopropyl phenol or propofol suitable for industrial or pharmaceutical use.

### BACKGROUND OF THE INVENTION AND PRIOR ART

2,6-diisopropylphenol, commonly known as “Propofol” is an intravenous anaesthetic agent known for its rapid onset and short duration of action. It plays a crucial role in medical procedures, providing anaesthesia for surgeries, sedation for critical care patients, and inducing unconsciousness swiftly with minimal side effects [Ref: Paul, E. M. et al.: Curr. Pharm. Des. 2004, 10, 3639-3649]. Its mechanism involves enhancing the inhibitory neurotransmitter gamma-aminobutyric acid (GABA) in the brain, leading to sedative and hypnotic effects [Ref: Sahinovic, M. M. et al.: Clinical Pharmacokinetics 57.12 (2018): 1539-1558.]. Propofol decreases cerebral oxygen consumption, reduces intracranial pressure, and has potent anticonvulsant, antioxidant, and anti-inflammatory properties.

Propofol is on the World Health Organization's List of Essential Medicines. Although shortages can occur with any drug, sterile injectable drugs such as propofol are particularly susceptible. Its importance became apparent during the peak of the COVID- 19 pandemic when demand for propofol exceeded supply. It should be noted that shortages also existed before the pandemic and were linked to quality issues that led to recalls and production delays [Ref: Jensen, V. Rappaport et. al.: New England Journal of Medicine, 363(9) (2010): 806-807.]. According to the Drug Shortage Program of the Food and Drug Administration (FDA), the shortage of Sterile Injectable Drugs has been about 50% over the past decade.

Early synthetic methods involved direct syntheses of propofol from phenol using propylene gas (a substitution with isopropanol) and a Lewis acid catalyst at high temperatures (300° C.) and pressures (3000 bar) [Ref: Kealy, T. J et al.: J. Org. Chem. 1961, 26, 987-992; U.S. Pat. No. 4,275,248A]. However, in accordance with the information as recited in U.S. Pat. No. 8,664,452, in addition to the high safety risks associated with the conditions, this route resulted in the formation of by-products such as 2,4- and 2,5-diisopropylphenol, 2,4,6-triisopropylphenol and others. The similarity in the boiling points of these impurities complicated the purification of propofol, hence, preventing propofol from fulfilling the regulatory standards for its medicinal applications. High-quality propofol synthesis at a commercial scale, had been reported by Bachem AG, wherein phenol was coupled with acetone using a strong base as a catalyst, albeit with minimum data in the literature. A more suitable strategy was found to use a protected 4-position of phenol such as 4-chlorophenol [Ref: Tsutsumi, S. et al.: Nippon Kagaku Zasshi 1956, 77, 737-738] or 4-hydroxybenzoic acid [Ref: Pramanik, C. et al.: Organic Process Research & Development 18.1 (2014): 152-156]. These starting materials were subjected to the alkylation process using isopropanol along with a strong acid giving an option for the in-situ generation of propylene. In a subsequent step, the chloride was removed by RaNi-catalyzed hydrogenation and the acid by decarboxylation under basic conditions to produce propofol. About a decade ago, several improvements were made in the process of obtaining propofol from 4-hydroxybenzoic acid, eliminating the disadvantages of exothermic neutralization steps with a phase separation followed by an extraction of the aqueous layer.

However, in the last few years, in order to develop more efficient ways of synthesizing propofol in terms of the number of unit operations, by-products, and temperature control, a specific focus has been made on the development of continuous flow chemical processes. Mougeot et al.: Molecules 26.23 (2021): 7183 recites the development of a continuous synthesis of propofol (in a milligram scale) using a flow chemistry process using 4-hydroxybenzoic acid and sulfuric acid. Later, Martins et al.: Journal of Flow Chemistry 12.3 (2022): 371-379 shows further enhancement in such continuous flow procedure by means of telescoped synthesis that produces tens of grams with a throughput of approximately 1 g/h. Furthermore, Vinet et al.: Organic Process Research & Development 26.8 (2022): 2330-2336 also showed a telescoped synthesis of propofol from starting material 4-hydroxybenzoate with the same throughput, but the yield was lower (73% in-solution, 46 to 49% isolated yield).

Recently, Sagandira, C. R. et. al., Sustainable Chemistry and Pharmacy, Vo. 42 (2024): 101793 reported microwave-assisted continuous flow synthesis of propofol using 4-hydroxybenzoic acid. By integrating microwave irradiation with flow technology for both steps, the overall residence time was reduced to 21 min and the throughput for the decarboxylation step reached up to about 7 g/h. In parallel with these academic efforts, commercial processes have also been disclosed in other patent literatures. One of the prior art processes as reported in US20230159420A1 describes a manufacturing and purification strategy for high-purity propofol that incorporates continuous steam distillation as an alternative to batch vacuum distillation, addressing purification challenges associated with closely related phenolic impurities. Additionally, WO2023111488A1 also reports an aqueous, pressurized decarboxylation process for converting 3,5-diisopropyl-4-hydroxybenzoic acid to propofol, with optional implementation under continuous conditions. However, all the above prior art processes require multiple stages of isolation and purification of the intermediate and the product, excessive impurity formation, long reaction times, use of toxic/flammable solvents etc.

Therefore, based on the knowledge acquired from the prior arts above, it has been inferred that there still remains a requirement in the art to develop an improved process for synthesizing propofol from substituted phenols by continuous flow chemistry. The improvement opportunities include a reduction in waste and number of purification steps, lesser usage of the acid equivalent, reduction in impurity formation, improvement in throughput, enhancement in process intensification, and modernization for scale-up.

### OBJECTIVES OF THE PRESENT INVENTION

An object of the invention is to overcome the disadvantages of the prior art.

Another object of the present invention is to provide a process for the preparation of a compound of formula (III) in a continuous manner with a high throughput of 11 g/h to 14 g/h or a space-time yield of 185 g·h⁻¹·L⁻¹ to 230 g·h⁻¹·L⁻¹.

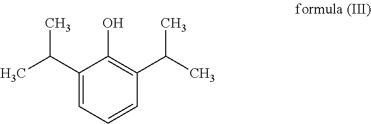

Yet another object of the present invention is to provide a process for the preparation of propofol in a continuous manner, followed by purification to obtain 98.0 to 99.4% purity and batch distillation to achieve 99.0-100% purity.

Yet another object of the present invention is to provide a continuous synthesis of propofol that results in a mass intensity of 58-70.

Yet another object of the present invention is to provide a process for the continuous synthesis of propofol, suitable for industrial-level scale-up.

### BRIEF SUMMARY OF THE INVENTION

An aspect of the present invention provides an industrially scalable process for the preparation of a compound of formula (III) in a continuous manner providing a throughput of 11-14 g/h

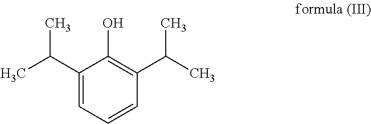

comprising the steps of:
a) alkylating a compound of formula (I)

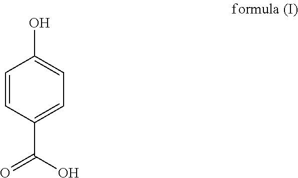

with an alkylating agent in water and in presence of an acid, wherein the volume ratio of alkylating agent:water:acid ranges between 1.66:0.10:5.8 and 3.00:0.25:9.70 to produce a compound of formula (II)

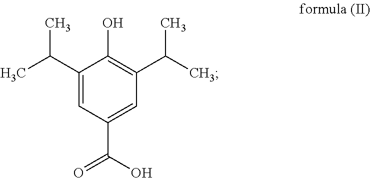 [SMILES: CC(C)c1cc(C(=O)O)cc(C(C)C)c1O]

b) decarboxylating the said compound (II) in presence of a base, an antioxidant and a solvent to produce the compound of formula (III).

Further benefits of this disclosure will be apparent to one skilled in the art.

### BRIEF DESCRIPTION OF THE ACCOMPANYING DRAWINGS

FIG. 1 (a, b, c, d, & e) graphically illustrates the effect of water (0 to 0.46 V) and 1.66 V IPA on the reaction kinetics for compound II synthesis in batch mode.

FIG. 2 (a, b, c) graphically illustrates the effect of the order of addition of reagents addition in step (a) of the currently developed process.

FIG. 3 (a) illustrates the currently developed synthetic scheme for Compound II by continuous flow chemistry; FIG. 3 (b) illustrates a comparative flow synthetic scheme for Compound II with different feeds.

FIG. 4 (a, b) illustrates the present continuous flow process steps (a) and (b) for synthesizing compound III (Propofol).

FIG. 5 (a) graphically illustrates the HPLC data for the said crude reaction mixture of step (b) which shows 94.1 A % purity; FIG. 5 (b) graphically illustrates the HPLC data for the crude product propofol (compound III) after purification which shows 97.6 A % purity; FIG. 5 (c) graphically illustrates the HPLC analysis data [that has been adjusted from a standard USP (United States Pharmacopeia)] for the final product propofol (compound III) after distillation which shows 99.9 A % purity.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that reference numbers are used to depict the same or similar elements, features, and structures.

### DESCRIPTION OF THE INVENTION

The following description with reference to the accompanying drawings is provided to assist in a comprehensive understanding of exemplary embodiments of the invention. It includes various specific details to assist in that understanding but these are to be regarded as merely exemplary.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings but are merely used by the inventor to enable a clear and consistent understanding of the invention. Accordingly, it should be apparent to those skilled in the art that the following description of exemplary embodiments of the present invention are provided for illustration purposes only and not for the purpose of limiting the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps, or components but does not preclude the presence or addition of one or more other features, integers, steps, components or groups thereof.

The present invention relates to a process for the preparation of a compound of formula (III) in a continuous manner as depicted in the following scheme A:

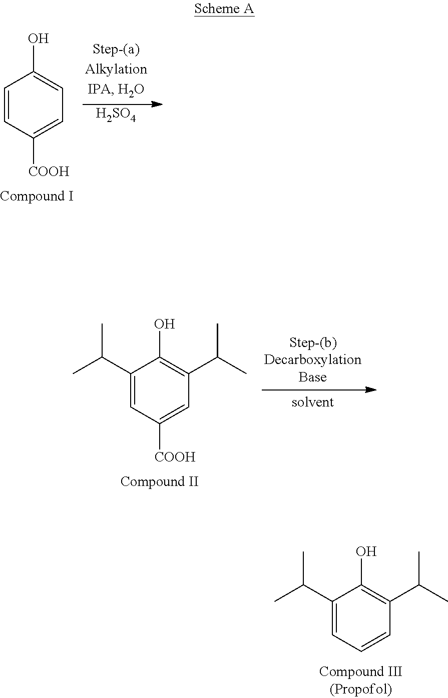

An embodiment of the present invention provides a process for synthesizing a compound of formula (III) in a continuous manner providing a high throughput of 11-14 g/h or a space-time yield of 185-230 g·h⁻¹·L⁻¹.

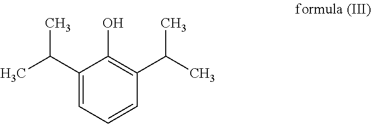

Another important embodiment of the present invention provides a process for the preparation of the said compound of formula (III) in a continuous manner using flow chemistry

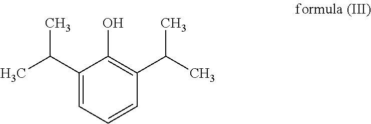

comprising the steps of:
a) alkylating a compound of formula (I)

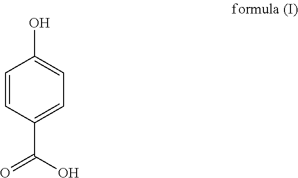

with an alkylating agent in water and in presence of an acid, wherein the volume ratio of said alkylating agent:water:acid ranges between 1.66:0.10:5.80 and 3.00:0.25:9.7 to produce a compound of formula (II)

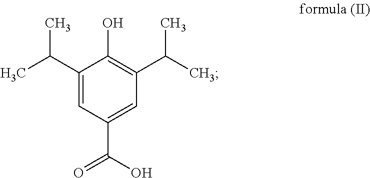 [SMILES: CC(C)c1cc(C(=O)O)cc(C(C)C)c1O]

b) decarboxylating the said compound (II) in presence of a base, an antioxidant and a solvent to produce the compound of formula (III).

In an aspect of the present invention, in step (a) of the currently developed process above, the starting material i.e. compound of formula I, specifically 4-hydroxybenzoic acid is found to provide a much cleaner profile and higher yield than the other commonly used starting materials known in the prior art such as 4-chlorophenol and 4-hydroxybenzoate respectively. The amount of 4-hydroxybenzoic acid used in the current process is 2.2M to 4.1M.

In another aspect of the present invention, in step (a) of the currently developed process above, the said alkylating agent used is selected from a group consisting of isopropyl alcohol, di-isopropyl ether, propylene and isopropyl halide, preferably isopropyl alcohol (IPA). The volume of IPA used is 1.66 V to 3.00 V.

A further aspect of the present invention is the use of water as a solvent in step (a) in a volume range of 0.10 to 0.25 V. It is known in the art that the presence of water within a defined range significantly reduces impurity formation during alkylation. On the other hand, the current inventors have observed that increasing the water content significantly reduces the solubility of the starting material, such as 4-hydroxybenzoic acid, especially in the IPA-water mixture. Thus, it has been found by the present inventors that maintaining a homogeneous reaction medium plays a critical role in a reliable continuous flow synthesis.

Therefore, in an embodiment of the present invention, to reach these effective volume ranges of water and IPA, their individual effects and relationships in view of the current reaction kinetics have been closely observed by the various experiments in batch (as illustrated in detail under the example 1 below). The results obtained shows that the best reaction outcome is achieved when the amount of water volume ranges between 0.10 V and 0.25 V and the volume of IPA ranges between 1.66 V and 3.00 V.

In another embodiment of the present invention, in step (a) the said acid used is selected as one or more from a group consisting of sulfuric acid, triflic, methanesulfonic, p-toluenesulfonic, phosphoric, and perchloric acid, preferably sulfuric acid. For the flow experiments, the stock solution (stream 1) of said 4-hydroxy benzoic acid is dissolved in said IPA:water (1.66:0.10 to 3.00:0.25) which results in a molar ratio ranging between 2.2 M and 4.1 M. Next, the said mixture is combined with H₂SO₄ (stream 2) to synthesize the compound of formula II, that produces a combined stream (stream 1 and stream 2) having a molarity of 0.65 to 0.80 M. Hence, it is evident from these experimental outcomes that by using the water and IPA volume ranges as obtained in the batch experiments, the equivalent of sulfuric acid (H₂SO₄) and residence times are significantly reduced compared to the previously reported conditions in flow chemistry. To reach these, residence time and H₂SO₄ equiv. relationships have been closely observed by conducting certain experiments in flow conditions (as illustrated in details under example 2 below). The equiv. of H₂SO₄ thus best suited for the currently developed flow process is chosen between 18 and 25 equiv. along with 1.5-2 equiv. of triflic acid [i.e. in volume range of 5.8 V to 9.70 V]. Such amount used is advantageously much lesser than those used in the processes as reported in the closest prior arts prior arts. Therefore, the currently developed process is not only economical but also, less hazardous and suitable for industrial level scale-up reactions.

In a further embodiment of the present invention, the process mass intensity (PMI) for the flow process is significantly improved taking the same organic stream to step (b) for decarboxylation. All the carryover impurities from step (a) are effectively purged out by the purification method in order to achieve 99.90-100% purity, thus, demonstrating the robustness of the present invention. It has thus been confirmed that the best reaction outcome is achieved by the currently developed process, wherein the alkylating agent (e.g. IPA), water, and acid (e.g. sulfuric acid) are used in a volume ratio ranging between 1.66:0.10:5.80 and 3.00:0.25:9.70, which provides a better outcome with respect to reaction kinetics than those reported in the prior arts.

In another embodiment of the present invention, it has been observed that the sequence of addition of all the above reagents such as alkylating agent, water, and acid in step (a) of the currently developed process has a significant impact on the outcome of the reaction kinetics towards the formation of compound II. Using the alkylating agent such as isopropyl alcohol (IPA) and water in a volume ratio of 6:94, the order of addition of reagents is studied. The results obtained shows that the reaction kinetics of current step (a), wherein before coming in contact with the compound of formula I, H2SO4 is preliminarily mixed with IPA for 20-30 mins, followed by the addition of water; such order of adding reagents surprisingly provides accelerated reaction kinetics for the compound of formula I within one hour. Eventually, it has been observed that after 3 hours, the proportion of compound II formed is around 80 A %.

In another embodiment of the present invention, in step (b) the base used is selected from a group consisting of n-butylamine (n-BuNH₂), imidazole, triethylamine (TEA), 1,8-diazabicyclo (5.4.0)undec-7-ene (DBU), N,N-Diisopropylethylamine (DIPEA), and isopropylamine, NaOH, K₂CO₃, potassium tert-butoxide, preferably n-BuNH₂. The said base is used in an amount ranging between 2 and 13 equivalent. More specifically, it has been found in the present flow reaction, that when n-BuNH₂ is used as a base in step (b), the reaction throughput is significantly improved to 11-14 g/h from the previously reported throughputs (around 3 g/h), along with higher isolated yield of 85 to 90% for the final product (formula III), thus achieving propofol with the desired high purity of 99.9 A % after distillation.

Furthermore, in another aspect of the current flow process, the solvent used in step (b) is chosen from 2-butoxyethanol, dimethylformamide (DMF), or toluene. Moreover, this step is carried out at a temperature range of 150° C. to 200° C.

Another important embodiment of the present invention provides an industrially scalable continuous flow synthesis of Compound II as described in Example 5 below. Reactions are performed on a Vapourtec R-series flow chemistry system using 1/16″ and 3/16″ O.D. PFA tubing is housed inside an insulated glass manifold for temperature control. Residence times are achieved through flow rates varying while maintaining the loop volumes constant. Only steady-state reaction streams are used to analyze in-process reaction profiles by LC. Purity and in-solution yield are determined by HPLC analysis (210 nm). These final set of reaction conditions thus provides i) 13.5 times higher throughput, ii) 0.72 vs 0.30 M process and iii) 2.3 times less sulfuric acid usage (compared to those amounts used by process disclosed in closest prior arts).

Yet another important embodiment of the present invention provides an industrially scalable continuous flow synthesis of compound III (Propofol) as described in Example 6 below. During the current synthesis, an antioxidant chosen from sodium metabisulfite, potassium metabisulfite, sodium bisulfite, or similar (0.5 to 3 mol %) is used as an additive to obtain the final colorless product of compound III (Propofol). It has thus been observed by the present invention that the preparation of propofol in a continuous manner, followed by purification provides 98.0 to 99.4% purity and batch distillation provides 99.0-100% purity.

The invention is now illustrated by way of non-limiting examples. The examples are intended to be purely exemplary of the invention, should therefore not be considered to limit the invention in any way.

### EXAMPLES

### Example 1: Individual Effects and Relationships of Alkylating Agents/Mixtures in View of the Current Reaction Kinetics in Batch Experiments

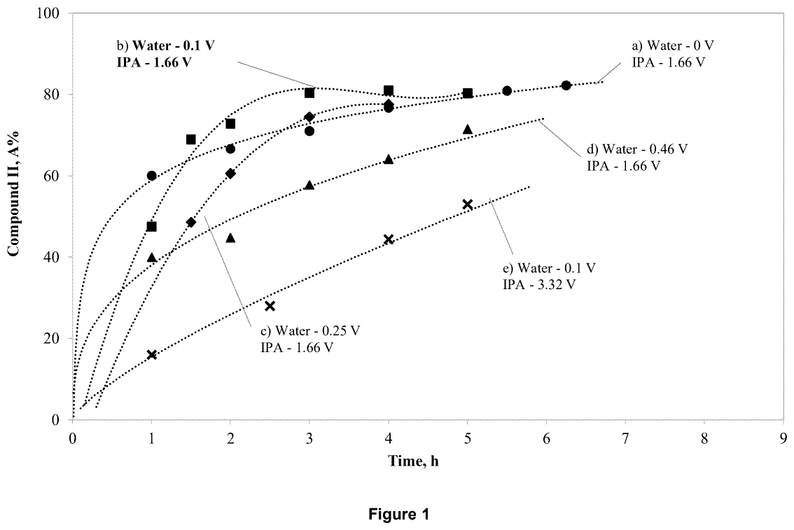

Example 1 below graphically illustrates the effective volume ranges of alkylating agents/mixtures such as water and IPA, their individual effects and relationships in view of the current reaction kinetics in batch.
Procedure: Concentrated sulfuric acid (9.3 equiv) is gradually added to a reaction vessel containing compound I i.e. 4-hydroxybenzoic acid (1 g), IPA (1.66 V) and water (from 0 to 0.46 V) pre-cooled to 0 to 5° C. The reaction mixture is then heated to 57-60° C. with kinetic monitoring via HPLC.
Results: The accompanying FIG. 1 shows the results obtained as below:
   i. FIG. 1 (a) graphically illustrates the reaction outcome without the addition of water (neat IPA) which has allowed the reaction to complete after 5 hrs,
   ii. FIG. 1 (b) graphically illustrates the reaction outcome on adding a small amount of water (0.1 V) which shows a slight reduction in the level of impurities and accelerates the kinetics at the initial stage.
   iii. FIG. 1 (c & d) graphically illustrates the reaction outcome when the same reaction (ii) is conducted with extended reaction time. It shows the formation of major by-products due to parallel and/or subsequent reactions with compound II;
   iv. FIG. 1 (e) graphically illustrates the reaction outcome of the addition of isopropanol (IPA) to water which shows a slowdown in the reaction rate.
Therefore, it is observed in the present invention that the best reaction outcome is achieved when the amount of water volume ranges between 0.10 V and 0.25 V and the volume of IPA ranges between 1.66 V and 3.00 V.

### Example 2: Relationship Between Residence Time and H₂SO₄ Equiv. Under Flow Conditions

Example 2 below illustrates the relationship between residence time and H₂SO₄ equiv. under flow conditions.
Procedure: Under the flow experiments, the stock solution (stream 1) of said 4-hydroxy benzoic acid is dissolved in said IPA:water (1.66:0.10 to 3.00:0.25) which results in a molar ratio ranging between 2.2 M and 4.1 M. Next, the said mixture is combined with H₂SO₄ (stream 2) to synthesize the compound of formula II, that produces a combined stream (stream 1 and stream 2) having a molarity of 0.65 to 0.80 M.
Results: Hence, with the water and IPA volume ranges as obtained in the batch experiments above, the equivalent of sulfuric acid (H₂SO₄) and residence times are significantly reduced compared to the previously reported conditions in flow chemistry. To reach these, residence time and H₂SO₄ equiv. relationships have been closely observed by the following experiments in flow conditions:
   a) Similar conversions previously reported the use of 41.25 equiv. H₂SO₄ and 30 min residence times at 60° C.; whereas, in the currently developed process the temperature is increased to 80° C. and the residence time is reduced to 10 min.
   b) Equivalence of H₂SO₄ is reduced to 18 to 25 equiv. and residence time is lowered to 5-10 min by increasing the temperature to 80 to 90° C.
   c) It has been further observed that such experimental conditions are only accessible through flow technology as otherwise longer residence time (typically observed in batch reactions) leads to impurity formation at such high temperatures. The flow outlet stream is thus continuously cooled down to minimize impurity formation.

Hence, the above critical parameters as obtained through batch reactions are similarly extrapolated to flow conditions in order to obtain the best results. The equiv. of H₂SO₄ used in the present flow process is finally chosen between 18 and 25 equiv. along with 2 equiv. of triflic acid. Such amount used is advantageously much lesser than those used in the processes as reported in the closest prior arts prior arts (as illustrated in detail in table 1 below). Therefore, the currently developed process is not only economical but also, less hazardous and suitable for industrial level scale-up reactions.

Furthermore, in the present invention, the process mass intensity (PMI) for the flow process is significantly improved taking the same organic stream to step (b) for decarboxylation. All the carryover impurities from step (a) are effectively purged out by the purification method in order to achieve 99.9% purity, thus, demonstrating the robustness of the present invention.

### Example 3: Comparable Data Between Reaction Kinetics or Parameters Achieved by the Currently Developed Process Vs. Those Reported in the Closest Prior Arts

Example 3 along with the following table 1 provides a comparable data that shows better reaction kinetics or parameters achieved by the currently developed process for synthesizing Compound II over those reported in the closest prior arts such as:
Reference A: Pramanik et al.: Organic Process Research & Development 18.1 (2014): 152-156
Reference B: Mougeot et al.: Molecules 26.23 (2021): 7183
Reference C: Martins et al.: Journal of Flow Chemistry 12.3 (2022): 371-379.
Reference D: Sagandira, C. R. et. al.: Sustainable Chemistry and Pharmacy, Vo. 42 (2024): 101793.

TABLE 1

| | | Water, | IPA:Water | H₂SO₄, | | Reaction | |
|---|---|---|---|---|---|---|---|
| Reference | IPA, V | V | ratio (by V) | equiv | Molarity | time, min | Yield, % |
| A | 1.66 | 0.25 | 87:13 | 9.28 | 1.31M | 90 | 84^{a} |
| | (3.0 equiv) | | (1.91 V) | (3.6 V) | | | |
| B | 3.30 | 3.30 | 50:50 | 75.50 | 0.20M | 40 | 84^{b} |
| | (6.0 equiv) | | (6.60 V) | (29.3 V) | | | |
| C | 6.65 | 1.20 | 85:15 | 41.25 | 0.30M | 30 | 85^{b} |
| | (12.0 equiv) | | (8.00 V) | (16.0 V) | | | |
| D | 4.0 | 1.00 | 4:1 | 23.20 | 0.52M | 20 | 86^{b} (93^{c}) |
| | (7.2 equiv) | | (5.00 V) | (9.0 V) | | | |
| Present | 1.66-3.00 | 0.10- | 94:7 | 18.00-25.00 | 0.72M | 5 | 80^{a} (90^{c}) |
| Invention | (3-5.4 equiv) | 0.24 | (1.8 V) | (5.8-9.7 V) | | | |
| | | | to 97:3 | | | | |
| | | | (5.50 V) | | | | |

Key:
^{a}Isolated by crystallization.
^{b}Isolated by silica gel column chromatography.
^{c}In-solution (HPLC) yield.

Hence, from the above comparable data, it is confirmed that the best reaction outcome is achieved by the currently developed process, wherein the alkylating agent (e.g. IPA), water, and acid (e.g. sulfuric acid) are used in a volume ratio ranging between 1.66:0.10:5.80 and 3.00:0.25:9.70, which provides a better outcome with respect to reaction kinetics than those reported in the prior arts.

### Example 4: Effect of Sequence of Addition of Reagents on the Outcome of the Reaction Kinetics Towards the Formation of Compound II

Example 4 below graphically shows that the sequence of addition of all the above reagents such as alkylating agent, water, and acid in step (a) of the currently developed process has a significant impact on the outcome of the reaction kinetics towards the formation of compound II. Using the alkylating agent such as isopropyl alcohol (IPA) and water in a volume ratio of 6:94, the order of addition of reagents is studied.

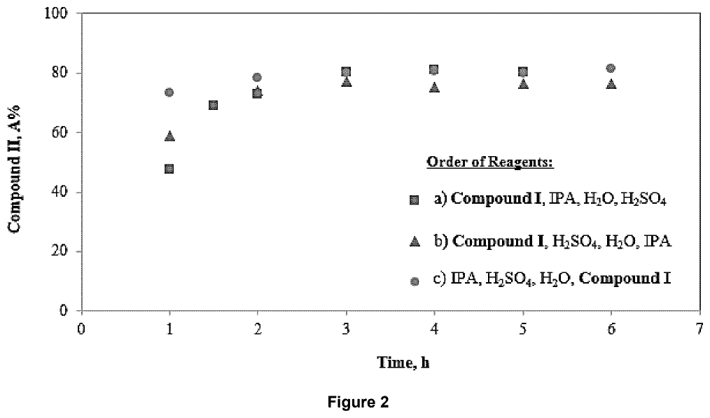

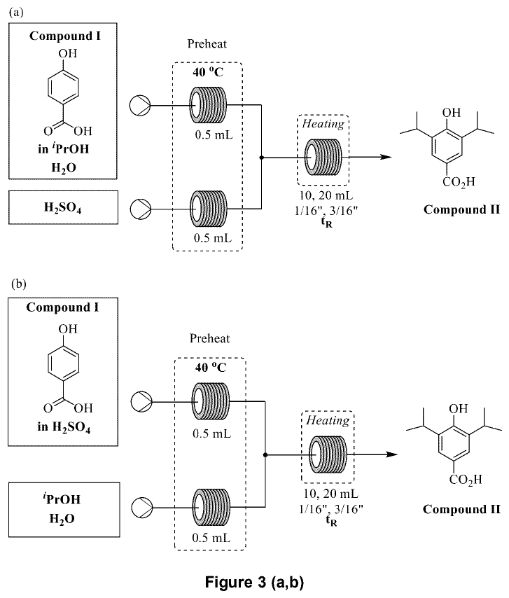

Results: The data obtained has been graphically represented in the accompanying FIG. 2 (a,b,c) as below:
i. FIGS. 2 (a) and 2 (b) graphically illustrate the reaction kinetics of current step (a), wherein the acid i.e. sulfuric acid (H₂SO₄) is added to a mixture of compound I, followed by IPA and water (in the said order) or IPA is added to compound I, sulfuric acid, followed by water (in the said order) respectively; both cases show the same result which is within the error.
ii. FIG. 2 (c) graphically illustrates the reaction kinetics of current step (a), wherein before coming in contact with the compound of formula I, H₂SO₄ is preliminarily mixed with IPA for 20-30 min, followed by the addition of water; such order of adding reagents surprisingly provides accelerated reaction kinetics for the compound of formula I within one hour. Eventually, it has been observed that after 3 hours, the proportion of compound II formed is around 80 A %.

### Example 5: An Industrially Scalable Continuous Flow Synthesis of Compound II

Example 5 below read along with the accompanying FIG. 3 (a,b) illustrates an industrially scalable continuous flow synthesis of Compound II.

### Procedure:

Currently Developed Continuous Flow Synthesis of 3,5-Diisopropyl-4-Hydroxybenzoic Acid (Compound II) [as Illustrated in Accompanying FIG. 3 (a)]:

A stock solution of Compound I (4-hydroxybenzoic acid) in IPA and water (Feed 1) is prepared by stirring until it completely gets dissolved at 50° C. and then allowed to cool to room temperature (RT). Concentrated sulfuric acid (18.0 equiv.) is prepared in a separate feed vessel (Feed 2). Feeds 1 and 2 are pumped by HPLC pumps at flow rates of 1.37 and 2.63 mL/min respectively. [as illustrated in accompanying FIG. 3 (a)]. Each feed is then fed into a 1/16″ O.D. pre-heating loop at 40° C. (residence time about one minute) and then combined in a T-mixer. Next, this reaction mixture stream is made to enter into a tubular reactor equipped with a full-length static mixer. The residence time in the reactor is 5 minutes at 90° C. After completion of the reaction, the reaction mixture is cooled to room temperature. A sample of the crude reaction mixture is next analysed by HPLC (230 nm) with water and acetonitrile (1:1 v/v) as diluent.

a Comparative Continuous Flow Synthesis of 3,5-Diisopropyl-4-Hydroxybenzoic Acid (Compound II) with Different Feeds [as Illustrated in Accompanying FIG. 3 (b)]:

A stock solution of Compound I (4-hydroxybenzoic acid) in concentrated sulfuric acid (Feed 1) is prepared. On the other hand, isopropyl alcohol (IPA) and water are prepared in a separate feed vessel (Feed 2) and stored at room temperature (RT) [as illustrated in accompanying FIG. 3 (b)]. Each feed is fed into a 1/16″ O.D. pre-heating loop at 40° C. (residence time about one minute) and then combined in a T-mixer. Next, the reaction mixture stream is made to enter a tubular reactor in order to react within a given time and at a given temperature. After completion of the reaction, the reaction mixture is cooled to room temperature. A sample of this crude reaction mixture is further analyzed by HPLC (230 nm) with water and acetonitrile (1:1 v/v) as diluent.

### Results:

The following results have been observed when read with the data illustrated in Table 2 below:

TABLE 2
Critical parameters for the synthesis of Compound II
under currently developed continuous-flow reactions

| | | | | | | | | HPLC ^{b} | |
|---|---|---|---|---|---|---|---|---|---|
| | | | | | | | | | In- |
| | Scale, | IPA, | Water, | H₂SO₄, | | T, ° | Throughput, | Comp. II, | solution |
| Entry | g | V | V | equiv | t_{R}, min | C. | g/h^{a} | A % | yield, % |
| 1^{c,e} | 0.9 | 6.80 | 1.20 | 41.25 | 30 | 60 | 1.65 | 83.4 | 94 (85^{h}) |
| 2^{c,g,e} | 0.9 | 6.80 | 1.20 | 41.25 | 30 | 40 | | | |
| 3^{c,e} | 0.9 | 6.80 | 1.20 | 41.25 | 15 | 80 | 3.30 | 74.0 | |
| 4^{c,e} | 0.9 | 6.80 | 1.20 | 41.25 | 10 | 80 | 4.95 | 78.6 | 93 |
| 5^{c,e} | 0.9 | 6.80 | 1.20 | 41.25 | 5 | 80 | 4.95 | 71.4 | |
| 6^{c,e} | 0.9 | 6.80 | 1.20 | 30.00 | 10 | 80 | 6.04 | 59.0 | |
| 7^{c,e} | 0.9 | 6.80 | 1.20 | 30.00 | 20 | 80 | 3.02 | 66.8 | |
| 8^{d,f} | 0.9 | 6.80 | 1.20 | 41.25 | 5 | 80 | 9.91 | 75.8 | |
| 9^{d,f} | 1.5 | 1.66 | 0.10 | 16.50 | 15 | 80 | 9.00 | 75.4 | |
| 10^{c,f} | 1.8 | 1.66 | 0.10 | 10.00 + | 20 | 70 | 8.15 | 79.5 | 93 |
| | | | | 2.00 | | | | | |
| | | | | Triflic | | | | | |
| | | | | acid | | | | | |
| 11^{d,f} | 20.0 | 1.66 | 0.10 | 12.00 | 5 | 90 | 34.87 | 79.6 | 93 |
| 12^{d,e} | 7.5 | 3.00 | 0.10 | 12.00 | 6 | 90 | 28.90 | 70.6 | 83 |
| 13^{d,e} | 2.5 | 3.00 | 0.10 | 15.00 | 5 | 90 | 25.34 | 72.1 | 86 |
| 14^{d,e} | 50.0 | 3.00 | 0.10 | 18.00 | 5 | 90 | 22.57 | 75.1 | 90 |

Key:
^{a}Reactions are performed on a Vapourtec R-series flow chemistry system using 1/16″ and 3/16″ O.D. PFA tubing is housed inside an insulated glass manifold for temperature control. Residence times are achieved through flow rates varying while maintaining the loop volumes constant. Only steady-state reaction streams are used to analyze in-process
reaction profiles by LC.
^{b} Purity and in-solution yield are determined by HPLC analysis (210 nm).
^{c}Two 1/16″ O.D. 10 mL coils are used as the reactor.
^{d} 3/16″ O.D. 20 mL coil with full-length static mixer is used as reactor.
^{e}Reaction performed according to flow schematic in accompanying FIG. 3 (a) and
^{f}Reaction performed according to flow schematic in accompanying FIG. 3 (b).
^{g}Line clogging is observed.
^{h}Isolated by crystallization.

### Observations:

A known prior art process (reference A of table 1 above) produced an in-solution yield of 94% for propofol (Table 2, entry 1).
At lower temperatures such as 40° C., reactor clogging is observed (Table 2, entry 2).
At higher temperatures, it is found that long reaction times resulted in an increase in by-products (Table 2, entries 3 and 4).
For 80° C., optimal conditions are achieved for 10 min versus 15 min (Table 2, entry 4 vs 3).
Unreacted starting material (Compound I) is observed with a further reduction of residence time (Table 2, entry 5).
Reduced kinetics and longer residence times are required when attempting to reduce the amount of sulfuric acid (Table 2, entries 6 and 7).
Improvements in conversion and profile are observed when intensifying the mixing of a relatively viscous reaction mass using a full-length static mixer installed in a 3/16″ PFR with a volume of 20 mL (Table 2, entry 8 vs 5).
A satisfactory reaction profile is achieved while using 1.66V IPA, 0.10V water, and 16 equivalent H₂SO₄ (Table 2, entry 9).
A comparably better reaction profile is further achieved by using 10 equivalent sulfuric acid with 2 equivalent triflic acid at 70° C. for 20 min (Table 2, entry 10). It is further observed that triflic acid significantly reduces the viscosity of the solution and a reactor without a static mixer is used.
Further, by increasing the temperature to 90° C., it is observed that a similar yield is achieved for 12 equivalent sulfuric acid at t_{R}=5 min (Table 2, entry 11).
The continuous flow synthesis is first performed at a 20 g scale, giving a throughput of 35 g/h (Table 2, entries 11). However, when the reaction is run at a 100 g scale, impurity is formed due to the stability of the starting material in H₂SO₄ [as illustrated in accompanying FIG. 3(a)].
Later, most importantly it has been found that for these set of reaction conditions, 12.0 and 15.0 equivalent sulfuric acid gives a low yield as 83% and 86%, while 90% yield is achieved for 18.0 equivalent sulfuric acid for a 50 g scale of synthetic (Table 2, entries 12, 13 and 14).
These final set of reaction conditions thus provides:
   13.5 times higher throughput (Table 2, entry 1 vs 14),
   0.72 vs 0.30 M process and
      2.3 times less sulfuric acid usage (compared to those amounts used by process disclosed in closest prior art reference A, B, C of Table 1 above).

### Example 6: An Industrially Scalable Continuous Flow Synthesis of Compound III

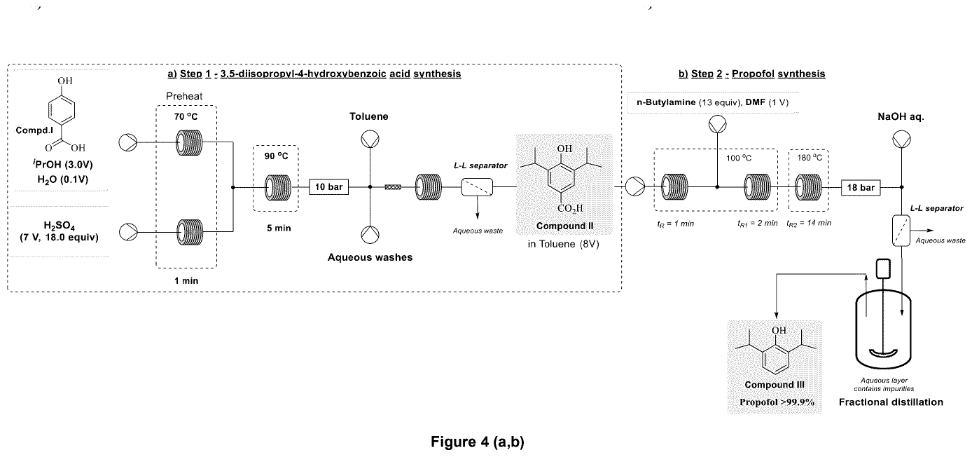

Example 6 below read along with the accompanying FIG. 4(a,b) illustrates an industrially scalable continuous flow synthesis of the final Compound III (Propofol):

### Procedure:

Step (a): Continuous Flow Synthesis of 3,5-Diisopropyl-4-Hydroxybenzoic Acid (Compound II) [as Illustrated in Accompanying FIG. 4(a)]
A stock solution of 4-hydroxybenzoic acid (compound I) (50 g, 1.0 equiv) in IPA (3.0 V) and water (0.1 V) (Feed 1) are stirred until completely dissolved at 50° C. and allowed to cool to room temperature (RT). Concentrated sulfuric acid (18.0 equiv.) is introduced in a separate feed vessel (Feed 2). Feeds 1 and 2 are pumped by HPLC pumps (Eldex) at flow rates of 1.37 and 2.63 mL/min. Each feed is fed into a 1/16″ pre-heating loop at 70° C. (residence time about one minute) and then combined in a T-mixer. Then the reaction mixture stream enters a 3/16″ 20 mL PFA tubular reactor equipped with a full-length polytetrafluoroethylene (PTFE) static mixer. The residence time in the reactor is 5 min at 90° C. After completion of the reaction, the reaction mixture is cooled to room temperature. A sample of the crude reaction mixture is analyzed by HPLC (210 nm) with water and acetonitrile (1:1 v/v) as diluent.
   The said crude reaction mixture is washed with NaOH (30%, 4 V) to pH 12, followed by layers cut. The aqueous layer is washed with toluene 4V followed by a phase cut. The aqueous layer is neutralized to pH 4-5 by HCl, in the presence of 6.35V toluene for extraction of compound II (purity 98.7% by HPLC 210 nm, excl. solvent) for 90% in-solution yield.
      Step (b): Continuous Flow Synthesis of 2,6-Diisopropylphenol (Propofol, Compound III) [as Illustrated in Accompanying FIG. 4(b)]
A stock solution of said 3,5-diisopropyl-4-hydroxybenzoic acid (compound II) in toluene [15.0 g in 6.35 V (157.5 mg/mL)] and DMF (1.0 V) is prepared in a feed vessel (Feed 1). Neat nBuNH₂ (13.0 equiv.) is prepared as a separate feed (Feed 2). Both stock solutions (stream 1 and stream 2) are sparged with nitrogen for 30 min continuously. Feeds 1 and 2 are then pumped by HPLC pumps (Eldex) at flow rates of about 2.02 and 1.41 mL/min respectively to the Vapourtec R-series flow chemistry system. Feed 1 is fed into a 1/16″ pre-heating loop (0.5 min, 100° C.) followed by a T-mixer with Feed 2 and a ⅛″ loop for gradual heating at 100° C. Streams with compound II and n-butylamine are fed into the ⅛″ reaction loop at 180° C. for 14 min reaction time with the BPR (about 18 bar). The pre-heating and reaction loops are made with a heat-tolerant material like stainless steel. The reaction mixture is cooled to room temperature and washed with HCl to attain pH 4-5, followed by a phase cut.
   The aqueous layer is then washed with toluene (2V) followed by separation and combining with the organic layer of the reaction mixture. The organic layer is washed with brine (20%, 4 V), saturated sodium bicarbonate (2×2 V), and water (1 V).
   0.5 mol % NaOH and 0.5 mol % sodium metabisulfite in 0.03 V water is also added to the reaction mixture. The mixture is then slowly heated to distill (2.0±0.3 mbar, 90-95° C. fraction) pure propofol as a colorless liquid (10.47 g, 87%) which helps to remove one major impurity (structure identified to be a dimer of compound II), thus obtaining compound III of 99.9% purity.
      During the current synthesis, an antioxidant chosen from sodium metabisulfite, potassium metabisulfite, sodium bisulfite, or similar (0.5 to 3 mol %) is used as an additive to obtain the final colorless product of compound III (Propofol).
Further analytical studies are conducted to understand the characteristics of this synthesized propofol

### Results:

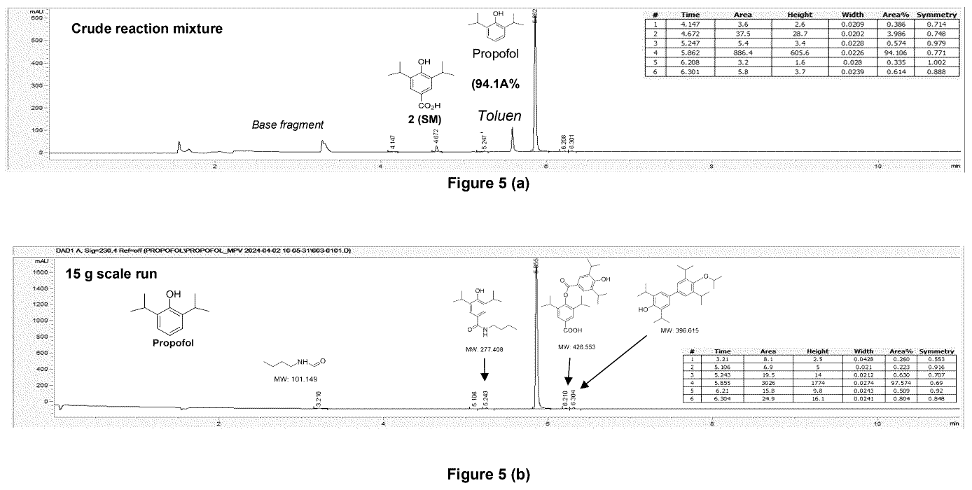

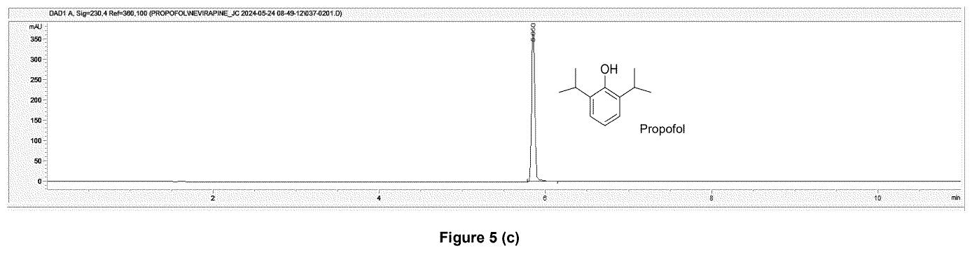

FIG. 5 (a) graphically illustrates the HPLC data for the said crude reaction mixture of step (b) which shows 94.1 A % purity;
FIG. 5 (b) graphically illustrates the HPLC data for the final product propofol (compound III) after purification which shows 97.6 A % purity;
FIG. 5 (c) graphically illustrates the HPLC data [that has been adjusted from a standard USP (United States Pharmacopeia)] for the final product propofol (compound III) after distillation which shows 99.9 A % purity.

### Technical Advantages

Technical advantages of the currently developed continuous flow process are provided below:
Propofol was synthesized in a continuous manner starting from 4-hydroxybenzoic acid, followed by batch distillation to achieve 99.9-100% purity.
Reduction in the amount of the major impurity

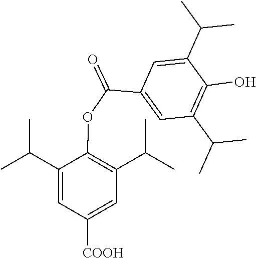 [SMILES: CC(C)c1cc(C(=O)Oc2c(C(C)C)cc(C(=O)O)cc2C(C)C)cc(C(C)C)c1O]

(structure identified to be a dimer of compound II) has been achieved without distillation, which is only 0.6 A %;
A high reaction throughput of 11-14 g/h is achieved;
The present process advantageously uses a very low amount of acid 18 to 25 equivalents, rendering the same economical and less hazardous, thus suitable for industrial usage.
The preheating of feed streams (with an alkylating agent, acid, and compound I) with gradual heating of the reaction mixture for the decarboxylation step (b) provides a more scalable, consistent, and reproducible continuous process.
   The currently developed continuous flow process is capable of producing highly pure propofol (up to 99.9%) with a space-time yield of approximately 185 to 230 g·h⁻¹·L⁻¹, enabling scalable production in continuous flow reactors without reliance on large reactor volumes. In contrast, previously reported batch processes typically require scale-up from approximately 100 L to >2000 L to achieve comparable output, which may impact reaction performance and controllability

What is claimed is:

1. An industrially scalable process for the preparation of a compound of formula (III) in a continuous manner providing a throughput of 11-14 g/h 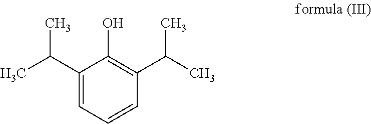
comprising the steps of:
a) alkylating a compound of formula (I) 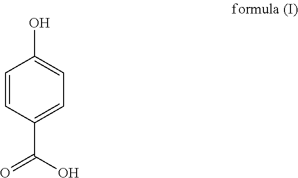
with an alkylating agent in water and in presence of an acid, wherein the volume ratio of alkylating agent:water:acid ranges between 1.66:0.10:5.8 and 3.00:0.25:9.70 to produce a compound of formula (II) 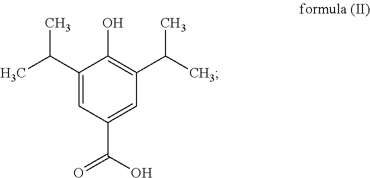 [SMILES: CC(C)c1cc(C(=O)O)cc(C(C)C)c1O]
and
b) decarboxylating the said compound (II) in presence of a base, an antioxidant and a solvent to produce the compound of formula (III).

2. The process as claimed in claim 1 of the present invention, wherein the starting material 4-hydroxybenzoic acid (compound I) is used in an amount ranging between 2.2 M and 4.1M.

3. The process as claimed in claim 1 of the present invention, wherein the said alkylating agent in step (a) is selected from a group consisting of isopropyl alcohol, di-isopropyl ether, propylene and isopropyl halide, preferably isopropyl alcohol (IPA).

4. The process as claimed in claim 3 of the present invention, wherein the said alkylating agent in step (a) is used in a volume range of 1.66 V to 3.00 V.

5. The process as claimed in claim 1 of the present invention, wherein water as a solvent in step (a) is used in a volume range of 0.10 to 0.25 V.

6. The process as claimed in claim 1 of the present invention, wherein the said acid used in step (a) is selected as one or more from a group consisting of sulfuric, triflic, methanesulfonic, p-toluenesulfonic, phosphoric and perchloric acid.

7. The process as claimed in claim 6 of the present invention, wherein said acid in step (a) is a combination of sulfuric acid and triflic acid.

8. The process as claimed in claim 7 of the present invention, wherein said sulfuric acid is used in an amount ranging between 18 and 25 equivalents.

9. The process as claimed in claim 7 of the present invention, wherein said triflic acid is used in an amount ranging between 1.5 and 2 equivalents.

10. The process as claimed in claim 1 of the present invention, wherein in step (b) the base used is selected from a group consisting of n-butylamine (n-BuNH₂), imidazole, triethylamine (TEA), 1,8-diazabicyclo(5.4.0)undec-7-ene (DBU), N,N-Diisopropylethylamine (DIPEA), and isopropylamine, NaOH, K₂CO₃, potassium tert-butoxide, preferably n-BuNH₂.

11. The process as claimed in claim 10 of the present invention, wherein in step (b) the base is used in an amount ranging between 2 and 13 equivalent.

12. The process as claimed in claim 1 of the present invention, wherein the solvent used in step (b) is selected from a group consisting of 2-butoxyethanol, dimethylformamide (DMF) and toluene.

13. The process as claimed in claim 1 of the present invention, wherein in step (b) the antioxidant used is selected from a group consisting of sodium metabisulfite, potassium metabisulfite, and sodium bisulfite.

14. The process as claimed in claim 13 of the present invention, wherein the antioxidant is used in an amount ranging between 0.5 and 3 mol %.

15. The process as claimed in claim 1 of the present invention provides compound III with 85 to 90% isolated yield.

16. The process as claimed in claim 1 of the present invention provides compound III with 99.90-100% purity.

17. The process as claimed in claim 1 of the present invention provides compound III with a space-time yield of 185-230 g·h⁻¹·L⁻¹.
